(19) United States
(12) Patent Application Publication
(10) Pub. No.: US 2015/0359835 A1
(43) Pub. Date: Dec. 17, 2015

(54) COMPOSITION OF A MEDICAL DEVICE OR COSMETIC PRODUCT BASED ON GRAPEFRUIT SEED EXTRACT, LADY'S MANTLE LEAF EXTRACT, STEVIA EXTRACT, AND CURCUMIN

(71) Applicants: Laurent Haddad, Neuilly-sur-Seine (FR); Vivian Stemmer, Paris (FR)

(72) Inventors: Laurent Haddad, Neuilly-sur-Seine (FR); Vivian Stemmer, Paris (FR)

(21) Appl. No.: 14/739,958

(22) Filed: Jun. 15, 2015

(30) Foreign Application Priority Data
Jun. 13, 2014 (FR) 1455409

(51) Int. Cl. A61K 36/752; A61K 36/28; A61K 9/00; A61K 36/73

(52) U.S. Cl. CPC A61K 36/752; A61K 36/73; A61K 36/28; A61K 9/006

(57) ABSTRACT

The present invention relates to a composition, especially in gel form, comprising at least one grapefruit seed extract, at least one lady's mantle leaf extract, at least one stevia extract, and at least curcumin. The invention also relates to said composition in the form of a medical device or of a cosmetic composition, and to the use thereof for the treatment of complaints of the buccal cavity. Moreover, the invention also pertains to the use of the composition for its analgesic activity. The invention also relates to the use of said composition for preserving the resident flora of the buccal cavity.

### CROSS-REFERENCE TO RELATED APPLICATION

This application claims the benefit of Application No. FR 1455409, filed Jun. 13, 2015, which is incorporated herein by reference.

The present invention relates to a composition comprising at least one grapefruit seed extract, at least one lady's mantle leaf extract, at least one stevia extract, and at least curcumin.

The invention also pertains to said composition in the form of a medical device or of a cosmetic composition, and also to the use thereof in the treatment of complaints of the buccal cavity, especially bleeding, lesions of the buccal mucosa and/or of the tongue, inflammatory disorders other than lesions, gingival and/or dental sensitivity, halitosis, dental eruptions, and bacterial, fungal, and viral infections.

The present invention also pertains to the fact that said composition is used for its analgesic effect and is therefore able to diminish and/or alleviate the pain associated with the above-described buccal cavity complaints.

The invention also relates to the use of said composition for preserving the resident buccal flora.

The buccal cavity is generally the site of multiple complaints, especially bleeding, inflammation such as gingivitis and periodontitis, which may in particular be caused by the bacterium Prevotella melaninogenica, lesions affecting the buccal mucosa and the tongue, such as mouth ulcers and other buccal ulcerations, and also bacterial or fungal infections (or buccal mycoses) such as thrush, and viral infections.

Indeed, mouth ulcers and other buccal ulcerations are lesions which affect the buccal mucosa and/or the tongue and which are usually manifested as swellings, sores or structural modifications of greater or lesser extent to the mouth, the tongue and the lips, and which may prove to be painful, unpleasant and an interference in terms of chewing, swallowing or phonation.

Ulcers typically appear preferentially at the gums, on the tongue and on the inside edge of the lips and cheeks, and often take the form of rounded or oval swellings covered with a whitish or yellowish membrane which may be surrounded by a reddish border. Ulcers may result from poor bucco-dental hygiene, from an allergy, from poor maintenance of dental prostheses, from a particular hormonal influence (menstrual periods or pregnancy), from vitamin deficiency, from the ingestion of certain foods such as nuts or Gruyere cheese, from certain drugs, from stress, or else from diseases which give rise to the appearance of ulcers, such as Behcet's disease, which are often viral in origin.

Other ulcerations frequently affecting the buccal mucosa and/or the tongue may be, for example, lesions which have a whitish appearance. Lesions of these kinds are often associated with an anomaly in keratinization, and may take on either on a benign character or a pre-cancerous character.

Among fungal infections, thrush is a buccal mycosis caused by the presence of a yeast known as Candida albicans. Thrush is most often manifested in the presence of plaques, which may be cream, whitish yellow or red in colour, situated on the tongue and on the buccal mucosa. Thrush is common in persons wearing, in particular, a dental appliance, in newborns, and also in persons having a weakened immune system.

Furthermore, the buccal cavity may also be the site of complaints linked essentially to bad breath (halitosis), to gingival and/or dental sensitivity, or else may be the location where numerous bacteria proliferate, such as Streptococcus mutans, which is the main cause of the appearance of caries.

In particular, gingival and/or dental sensitivity may occur especially in the event of retraction of the gums around the teeth or of loss of gum tissue. For example, the loss of gum tissue may come about subsequent to excessive brushing of teeth or else an absence of brushing and of regular use of dental floss. This loss leads to the root of the tooth being exposed, along with the canals connecting the root and its nerve centre. Accordingly, this loss will give rise to pain following a thermal stimulus, in other words when the teeth and/or gum make contact with a hot or cold liquid.

Similarly, Streptococcus mutans is a bacterium which adheres strongly to dental tissues and to the other bacteria present in the buccal cavity. This bacterium has a tendency to produce a polysaccharide biofilm on the teeth or a dental plaque, which may lead to the appearance of caries.

These different types of disorders of the buccal cavity of course induce uncomfortable sensations and/or pain in human beings.

In response to this, numerous pharmaceutical and/or cosmetic compositions, for rinsing or otherwise, which are intended for bucco-dental hygiene have been developed in the prior art for treating these various types of disorders, in order to reduce or eliminate the sensations of discomfort and/or the associated pain. However, the presently available bucco-dental hygiene compositions still have a number of drawbacks.

This is because such compositions often have pH values which are too acidic relative to the pH value in the buccal cavity, which is generally neutral, owing to the abundant presence of saliva.

Moreover, these compositions frequently give rise to more or less acute pain in the user when they come into contact with the buccal mucosa and/or the tongue, especially when they are applied to bleeding, lesions or plaques caused by fungal infections.

Lastly, these compositions are usually difficult to apply, especially to sores and/or lesions, owing to the aqueous physiological state of the buccal cavity. The reason is that the saliva tends to prevent the composition becoming affixed on the surface to be treated, which has the effect of reducing the efficacy of that composition. In other words, such compositions do not adhere sufficiently to the buccal mucosa and/or to the tongue, and have a tendency to be removed too rapidly before being able to exert their full effect. This absence of adhesion is observed in particular in bucco-dental hygiene compositions for use without rinsing.

There is therefore a genuine need to provide compositions which are capable in particular of effectively treating the aforementioned buccal cavity disorder or disorders while having a pH which is close to that of the saliva, and minimizing the pain that is felt at the time when they are applied.

There is also a genuine need to provide compositions which are capable, moreover, of adhering readily to the various locations in the buccal cavity that are to be treated.

In light of the above, the present invention particularly provides a composition comprising i) at least one grapefruit seed extract, ii) at least one lady's mantle leaf extract, iii) at least one stevia extract and iv) at least curcumin.

The composition according to the invention therefore comprises a synergistic combination of pleiotropic substances in order to obtain the properties described.

The composition according to the invention does in fact allow effective treatment of the buccal cavity disorders as described above, while respecting the pH in the buccal cavity. More particularly, the pH of the composition varies from 6.8 to 7.8, preferably from 7.3 to 7.7.

More particularly, the composition is not an irritant in the skin sense, allowing it to combine harmlessness with effectiveness.

Even more particularly, the application of the composition according to the invention to the surfaces of the buccal cavity that are to be treated gives rise to reactions which are markedly less painful than the conventional compositions.

Furthermore, the composition according to the invention also has the advantage that when applied, it is able to form a film capable of adhering to the surface of the various locations in the buccal cavity that are to be treated, especially on the buccal mucosa and on the tongue.

The adhesion of the film thus formed is promoted by the aqueous physiological state of the buccal cavity, by virtue of a thickening system.

The adhesion of the film extends the time of action of the composition on the surface to be treated. Expressed alternatively, the composition according to the invention is not eliminated too rapidly from the buccal cavity.

The composition according to the invention therefore produces an enhanced bioadhesive effect at the surface to be treated within the buccal cavity.

In the sense of the present invention, the “bioadhesive” character denotes the property of a composition to adhere to a biological tissue, especially a mucosa in the buccal cavity, more particularly to the epithelium of the mucosa, in order to create a new interface on the mucosa and to maintain itself thereon for a longer or shorter time. The mechanism of the bioadhesive effect is described in the scientific literature.

The bioadhesive effect allows the composition to be used effectively without rinsing.

The composition according to the invention has the advantage of being in accordance with Regulation (EC) 1223/2009.

However, given that the composition will be in contact with the buccal mucosa, the choice has been made to select only cosmetic raw materials which:
have a food or pharmaceutical grade,
are composed of substances authorized as food additives by Regulation 1129/2011 or which have a history of use in the food industry.

Moreover, the composition according to the invention has the advantage of being able to spread over all the entire buccal cavity surface or surfaces to be treated, thereby helping to improve its efficacy. For example, the composition is able to spread easily over all of the lesion to be treated, thereby promoting its scarring.

The composition of the present invention therefore has a good wetting and moisturizing capacity.

The composition according to the invention therefore has the advantage of effectively treating complaints of the buccal cavity and of diminishing and/or alleviating the pain associated with such complaints.

Especially when the buccal cavity is the site of complaints, the composition according to the invention may be applied directly to the surface or surfaces to be treated, more particularly to the lesion or lesions affecting the buccal mucosa and/or the tongue, in order to allow scarring thereof and to reduce the unpleasant sensations of pain and/or of discomfort caused by such complaints.

Accordingly, the composition of the invention also has an analgesic activity, which is realized by protection against tissue attack and saturation of the nociceptors.

The composition according to the invention also has the advantage of effectively treating buccal bacterial, fungal or mycotic infections such as thrush, and viral infections.

More particularly, when the buccal cavity is the site of fungal infections such as thrush, the composition according to the invention may be applied directly to the surface or surfaces to be treated in order to attenuate and/or eliminate the yeast named Candida albicans which is responsible for this infection, and also its proliferation.

In other words, the composition according to the invention has the advantage of exhibiting antimycotic properties while maintaining the resident buccal flora.

Furthermore, the composition according to the invention has the advantage of alleviating the discomfort associated with gingival and/or dental sensitivity, and also the proliferation of bacteria such as Streptococcus mutans, Prevotella melaninogenica, Pseudomonas aeruginosa, Staphylococcus aureus, and Escherichia coli.

Accordingly, when the buccal cavity is the location of proliferation of bacteria such as Streptococcus mutans, Prevotella melaninogenica, Pseudomonas aeruginosa, Staphylococcus aureus, or Escherichia coli, the composition according to the invention may be applied directly to the surface or surfaces to be treated in order to reduce the proliferation of the bacteria.

The composition according to the invention has the advantage in particular of exhibiting antimicrobial properties while maintaining the resident buccal flora.

The composition according to the invention has the advantage in particular of exhibiting antiviral properties with respect to the herpes simplex virus (HSV) which is responsible for buccal herpes.

Moreover, by virtue of its water-soluble character, the composition may be inserted into various bucco-dental hygiene compositions, particularly into dentifrices and mouthwashes.

The result is that the composition according to the invention has the advantage that it can be used in the therapeutic sector, particularly for treating buccal cavity complaints or different complaints, and also in the cosmetic sector, especially for cosmetic treatment of the buccal cavity.

The present invention likewise relates to the composition as described above in the form of a medical device.

The invention also pertains to the composition as described above in the form of a cosmetic composition.

Similarly, the invention likewise provides the composition as described above for use in the therapeutic sector.

More particularly, the invention pertains to the composition for use in the treatment of buccal cavity complaints.

Another subject of the present invention relates to the composition as described above for use in the diminishment or alleviation of the pain caused by buccal cavity complaints.

The invention also pertains to the use of the composition as described above for preserving the resident flora of the buccal cavity.

In accordance with the present invention, in the text below, unless otherwise indicated, the end points of a range of values are included in that range.

Other features and advantages of the invention will emerge more clearly from a reading of the description and examples which follow.

As indicated above, the composition according to the invention comprises i) at least one grapefruit seed extract.

The grapefruit extract used in the composition according to the invention essentially comprises flavonoids and ascorbic acid, optionally glycerol, grapefruit seed or peel extracts, and a blue agave extract.

The grapefruit seed extract used in the composition according to the invention preferably has a food or pharmaceutical grade.

The composition according to the invention preferably comprises the grapefruit seed extract in an amount of from 1% to 40% by weight, more preferably in an amount of from 10% to 35% by weight relative to the total weight of the composition.

As indicated above, the composition according to the invention also comprises ii) at least one lady's mantle leaf extract.

The lady's mantle leaf extract used in the composition according to the invention is preferably obtained from an extraction process in which dry lady's mantle leaves, ground or pulverized, are subjected to extraction with a mixture comprising water and alcohol.

The extract is preferably made from 170 g of dried leaves per kg of final extract.

The extraction may optionally take place with stirring at a temperature of from 10° to 30° C. for a time which may be from 8 to 20 hours.

The lady's mantle leaf extract used in the composition is preferably an extract of common lady's mantle leaf (or Alchemilla vulgaris L).

The lady's mantle leaf extract is preferably a composition which may contain from 1.5% to 100% by weight of lady's mantle leaf.

More particularly, the lady's mantle leaf extract which can be used in the composition of the invention may be an aqueous-alcoholic solution based on common lady's mantle leaf extract.

The composition according to the invention preferably comprises the lady's mantle leaf extract in an amount of from 1 to 10% by weight, more preferably in an amount of from 4 to 6.5% by weight relative to the total weight of the composition.

As indicated above, the composition according to the invention also comprises iii) at least one stevia extract.

The stevia extract used in the composition according to the invention is preferably obtained from a process of extraction with demineralized water, in which the aerial parts (leaves) of stevia, which are fresh or dry, ground or pulverized, are subjected to extraction.

The extraction may take place optionally with stirring at a temperature of from 95° C. to 99° C. for a time which may be up to 4 hours.

The solutions obtained following the extraction may optionally be concentrated and/or oven-dried under vacuum and cooled to ambient temperature.

The extraction process may comprise a crystallizing step after the drying step, with the aid of a solvent based on ethanol and water.

The stevia extract is preferably an extract of Stevia rebaudiana, especially a Stevia rebaudiana leaf extract.

The stevia extract preferably used in the composition according to the invention is a Stevia rebaudiana extract sold under the trade name Stevia extract 97% REB-A by Quimdis.

The composition according to the invention preferably comprises the stevia extract in an amount of from 0.025% to 0.1% by weight, more preferably in an amount of from 0.045% to 0.065% by weight relative to the total weight of the composition.

As indicated above, the composition according to the invention also comprises iv) at least curcumin.

Curcumin [1,7-bis-(4-hydroxy-3-methoxyphenyl)-1,6-hepta-diene-3,5-dione] is a hydrophobic polyphenol derivative derived from the spice turmeric.

The composition according to the invention preferably comprises the curcumin in an amount of from 0.0001% to 0.01% by weight, more particularly from 0.0001% to 0.001% by weight, relative to the total weight of the composition.

According to one embodiment of the invention, the composition comprises at least one grapefruit seed extract, at least one common lady's mantle leaf extract (or Alchemilla vulgaris L extract), at least one Stevia rebaudiana leaf extract and at least curcumin, in particular curcumin at 90%.

The composition according to the invention preferably comprises at least one gelling agent.

The composition according to the invention is therefore present advantageously in the form of a gel, more particularly a gel which is used without rinsing, thereby facilitating its attachment and its application and its adhesion to the surface or surfaces to be treated within the buccal cavity.

The gelling agent makes it possible in particular to enhance the adhesion of the composition to the surface to be treated in the buccal cavity, and to delay its dissolution. The activity time of the composition is consequently prolonged, thereby enhancing its efficacy.

By virtue of its pseudoplastic behaviour, the gelling agent also makes it possible to:
reduce the surface tension of the composition according to the invention relative to the surface to be treated in the buccal cavity, when it is subjected to a physical stress, thereby leading to an improvement in its spreading.
increase the surface tension of the composition according to the invention relative to the surface to be treated in the buccal cavity when the composition is not subject to any physical stress, thereby allowing its bioadhesiveness to be enhanced.

The composition according to the invention preferably comprises at least one nonionic gelling agent.

A gelling agent in the sense of the present invention means an agent capable of thickening the composition according to the invention.

The viscosity of the composition is preferably measured by means of a Brookfield MV-III rheometer at ambient temperature and at a shear rate of 10 rpm.

The nonionic gelling agent is preferably selected from polysaccharides, more particularly xanthan gums, guar gums, alginates and polymers of celluloses such as hydroxyethylcellulose.

The composition according to the invention more preferably comprises at least one xanthan gum.

The gelling agent is preferably present in the composition according to the invention in an amount of from 1% to 2% by weight and more particularly in an amount of from 1% to 1.3% by weight relative to the total weight of the composition.

The composition according to the invention may advantageously further comprise sodium hyaluronate.

Sodium hyaluronate promotes the formation of a film on the surface to be treated in the buccal cavity.

Sodium hyaluronate may preferably be present in the composition according to the invention in an amount of from 0.1% to 1.5% by weight and more particularly in an amount of from 0.85% to 1.1% by weight relative to the total weight of the composition.

According to one preferred embodiment, the composition is in the form of a gel and comprises at least one grapefruit seed extract, at least one common lady's mantle leaf extract (or Alchemilla vulgaris L extract), at least one Stevia rebaudiana leaf extract, at least curcumin and at least one nonionic gelling agent.

In accordance with this embodiment, the nonionic gelling agent is selected in particular from polysaccharides.

More particularly the nonionic gelling agent is a xanthan gum.

In accordance with this embodiment, the composition further comprises sodium hyaluronate.

According to one embodiment, the composition may also comprise at least one plant extract other than the plant extracts described above.

More particularly, the plant extract is preferably a clove extract, especially an essential oil of cloves.

The plant extract may preferably be present in the composition according to the invention in an amount of from 0.05% to 1% by weight and more preferably in an amount of from 0.25% to 0.5% by weight relative to the total weight of the composition.

The composition according to the invention may also comprise at least one oil, especially an oil of plant origin.

An oil is any non-aqueous liquid compound which is insoluble in water at ambient temperature (25° C.) and at atmospheric pressure (760 mmHg).

More particularly, the oil of plant origin that can be used in the composition may be avocado oil.

The oil of plant origin may preferably be present in the composition according to the invention in an amount of from 0.1% to 1% by weight and more preferably in an amount of from 0.35% to 0.60% by weight relative to the total weight of the composition.

The composition according to the invention may further comprise one or more additives selected from nonionic surfactants, anionic and nonionic polymers or mixtures thereof, essential oils, anti-inflammatories, whiteners, agents to counter dental plaque, such as cetylpyridinium chloride, and dispersants; film formers, antioxidants, sweeteners, antifungals and antibacterials, calmatives, moisturizers and humectants, anti-halitosis agents, astringents, anti-oedema agents; agents which promote cell growth; agents with a barrier effect to rubbing and to acidic pH.

The composition according to the invention has in particular a pH of from 6.8 to 7.8, preferably from 7.3 to 7.7.

The composition according to the invention may preferably further comprise one or more cationic surfactants, more particularly cetylpyridinium chloride.

According to one particular embodiment, the composition comprises:
at least one grapefruit seed extract,
at least one lady's mantle leaf extract (or Alchemilla vulgaris L extract),
at least one Stevia rebaudiana extract,
at least curcumin,
at least one nonionic gelling agent, more particularly xanthan gum,
hyaluronic acid.

A further subject of the present invention is the composition as described above in the form of a medical device.

More particularly, the composition according to the invention takes the form of a medical device as defined according to Directive 2007/47/EC.

According to one embodiment, the medical device comprises the composition according to the invention.

The invention further relates to the composition as described above for use in the therapeutic field.

More particularly, the invention further relates to the composition as described above for use for the treatment of complaints of the buccal cavity.

Thus the invention also pertains to a method for treating complaints of the buccal cavity, which comprises applying the composition as described above to the surface or surfaces of the buccal cavity that are to be treated.

For the purposes of the present invention, the buccal cavity is delimited by the palette, the floor of the mouth, the cheeks, the lips and also the uvula and the palatine arches on either side of the uvula.

More particularly still, the composition according to the invention is used for the treatment of buccal cavity complaints that may be caused by tooth and/or gum care.

“Tooth and/or gum care” in the sense of the present invention refers to care of the teeth and/or gums that is employed for the purpose of bucco-dental hygiene and is likely to give rise, in particular, to bleeding, lesions, more particularly ulcers and ulcerations within the buccal cavity, or else inflammatory disorders.

Thus the tooth and/or gum care may be, for example, non-surgical bucco-dental hygiene care, such as scaling, or tooth brushing that causes inflammation or bleeding of the gums.

The tooth and/or gum care may also correspond to surgical interventions on the teeth and/or the gums, for example a dental implant, the installation of a crown or a bridge, or a surgical operation aimed at treating the gums that may be affected by a periodontal disease. Generally speaking, bleeding may be observed following surgical interventions.

The composition according to the invention may therefore be applied by a user after dental care, or by a dentist on his or her care interventions.

The composition according to the invention is preferably used for treating buccal cavity complaints selected from bleeding, lesions of the buccal mucosa and/or of the tongue, inflammatory disorders other than lesions, dental eruptions, gum sensitivity, tooth sensitivity, halitosis, bacterial and fungal infections, or mycoses and viral infections.

According to one embodiment, the composition according to the invention is used for the treatment of bleeding of the buccal cavity.

Thus the composition of the invention may be used for treating bleeding of the buccal cavity, especially the bleeding caused by the chewing of certain foods, periodontal diseases, gum and/or tooth care such as scaling, brushing or surgical interventions.

According to one preferred embodiment, the composition according to the invention is used for the treatment of lesions of the buccal mucosa and/or of the tongue.

The “buccal mucosa” in the sense of the present invention means the mucous membrane which covers the cheeks and the gums.

The composition according to the invention has the advantage in particular of being able to form a film that is able to adhere properly to the lesion of the buccal mucosa and/or of the tongue, thereby improving the healing thereof. Accordingly, the composition forms a protective film between the lesion to be treated and the saliva, which promotes an analgesic effect.

In accordance with this embodiment, the composition according to the invention is used more specifically still in the treatment of mouth ulcers.

“Mouth ulcers” in the sense of the present invention mean lesions which affect, in particular, the buccal mucosa and which appear on the gums, on the tongue and also on the inside edge of the lips and the cheeks. In the sense of the invention, the term “ulcer” encompasses both miliary ulcers, which are ulcers having a diameter of less than one centimetre, and giant ulcers, which are ulcers with a diameter of more than one centimetre.

According to one preferred embodiment, the composition of the invention is used for the treatment of inflammatory disorders other than lesions.

The inflammatory disorders correspond more particularly to irritation and/or burns to the buccal mucosa, especially the gums.

Inflammatory disorders may result in particular from gingivitis, which has a tendency to irritate and inflame the gums.

Inflammatory disorders are also likely to appear following surgical interventions which may, for example, give rise to oedemas.

Thus the composition according to the invention is used for the treatment of irritation, burns and swelling of the gums.

According to another embodiment, the composition of the invention is used for the treatment of gum sensitivity, tooth sensitivity and dental eruptions.

In particular, the composition according to the invention allows a reduction in the discomfort associated with gum sensitivity and/or tooth sensitivity, especially the discomfort felt when the teeth and/or the gums are in contact with a medium, more particularly a liquid, which is cold or hot.

In the sense of the present invention, discomfort associated with gum and/or tooth sensitivity refers to discomfort felt in the gum and/or teeth following a thermal stimulus, in other words contact of the teeth and/or gums with a hot or cold medium, or following mechanical stimulus such as tooth brushing.

The composition according to the invention allows a reduction in particular in the discomfort associated with gum and/or tooth sensitivity caused by tooth brushing.

Furthermore, the composition also allows a reduction in discomfort in the case of dental eruption.

According to another embodiment, the composition according to the invention is used for the treatment of bacterial infections.

The invention preferably pertains to a composition as described above for use in the treatment of complaints caused by bacteria selected from Streptococcus mutans, Prevotella melaninogenica, Pseudomonas aeruginosa, Staphylococcus aureus, and Escherichia coli.

The composition is used more preferably in the treatment of bacteria selected from Streptococcus mutans, Prevotella melaninogenica, Pseudomonas aeruginosa, Staphylococcus aureus, and Escherichia coli.

Thus the composition is used in particular to reduce the proliferation of bacteria selected from Streptococcus mutans, Prevotella melaninogenica, Pseudomonas aeruginosa, Staphylococcus aureus, and Escherichia coli.

According to another preferred embodiment, the composition according to the invention is used for the treatment of fungal infections.

The composition according to the invention is preferably used for the treatment of fungal infections caused by the presence of a yeast called Candida albicans or a fungus called Aspergillus brasiliensis.

In other words, the composition according to the invention may be used for the treatment of thrush.

More particularly, the composition according to the invention is used to reduce the proliferation of Candida albicans or of Aspergillus brasiliensis.

According to another embodiment, the composition according to the invention is used for the treatment of viral infections, more particularly against herpes virus.

In other words, the composition according to the invention is used for the treatment of complaints linked to the presence of pathogenic microorganisms in the buccal cavity.

The composition according to the invention may be used in the treatment of one or more complaints as described above, particularly lesions of the buccal cavity, microbial infections and halitosis.

The composition may therefore be used in the treatment of a number of complaints simultaneously.

The composition according to the invention is more preferably used for the treatment of buccal cavity complaints selected from bleeding, mouth ulcers, gum sensitivity, halitosis, microbial infections, especially bacterial and fungal infections or mycoses and viral infections.

Thus the composition according to the invention makes it possible to diminish or even eliminate the pain associated with buccal cavity complaints, by virtue of its analgesic effect.

In other words, the invention also relates to a composition used for the treatment or alleviation of the pain associated with the buccal cavity complaints as described above.

This is because the composition is able to generate an analgesic process by protection from tissue attack and saturation of the nociceptors.

Expressed alternatively, the invention also relates to a composition according to the invention that is used for its analgesic effect in association with buccal cavity complaints.

Furthermore, the composition according to the invention may be used in a therapeutic field other than that of the dental sector.

More particularly, the invention aims to provide a composition as described above for use in the treatment of complaints other than those of the buccal cavity.

More particularly, the composition as described above may be used in the treatment of complaints other than those described above, in particular non chronic corporal wounds, namely wounds which are intended to be healed in a time limit ranging from 4 to 6 weeks after their apparition.

According to one particular embodiment, the invention relates to the in vitro use of the composition as described above against the proliferation of microorganisms selected from Pseudomonas aeruginosa, Staphylococcus aureus, Escherichia coli, Candida albicans, and Aspergillus brasiliensis.

The present invention also relates to the composition as described above in the form of a cosmetic composition.

This is because the composition according to the invention has the advantage of being able to treat the buccal cavity cosmetically.

More particularly, the composition allows protection of the buccal mucosa.

Moreover, the invention provides the use of the composition according to the invention for the preservation of the resident flora of the buccal cavity.

In other words, the invention comprises using the composition as described above to maintain the equilibrium of the resident buccal flora.

The composition according to the invention is preferably used for cleansing the buccal cavity, since it has advantageous antimicrobial activity while respecting the resident buccal flora.

According to one embodiment, the composition is applied to the surface of the buccal cavity that is to be treated.

The composition may in particular be applied during a period of three weeks every two months, twice per day.

The composition according to the invention is preferably applied to the lesions of the buccal mucosa and/or of the tongue, especially to ulcers.

The composition according to the invention may be packaged in a pump flask.

The composition according to the invention has the advantage in particular that it can be introduced into numerous buccodental hygiene compositions such as dentifrices, compositions intended for the polishing and for the gloss of the enamel, and mouthwashes.

The composition according to the invention is preferably incorporated in particular into dentifrices and mouthwashes.

The examples which follow serve to illustrate the invention but without having any limitative character.

### EXAMPLES

### Example I

Compositions (A) and (B) are prepared, the amounts therein being expressed as percentages by weight unless otherwise indicated.

| | Composition | Composition |
|---|---|---|
| | A | B |
| Common lady's mantle extract | 5 | 5 |
| Grapefruit seed extract | 40 | 40 |
| (Citrus paradisi peel extract) | | |
| Stevia rebaudiana extract | 0.05 | 0.05 |
| Curcumin | 0.001 | 0.001 |
| Clove extract | — | 1 |
| (Eugenia caryophyllus bud oil) | | |
| Avocado oil | — | 0.3 |
| (Persea gratissima oil) | | |
| Sodium bicarbonate | 5 | — |
| Alcohol | 4 | — |
| Water | qs 100 | qs 100 |

The pH of these preparations is between 6.8 and 7.8.

These preparations are found to be capable of application without causing pain in the user.

### Example II

### I. Preparation of Composition (C) According to the Invention

Composition (C) according to the invention is prepared in accordance with the procedure described below. The amounts are expressed as percentages by weight unless otherwise indicated.

| | Amounts (% by weight) | |
|---|---|---|
| Xanthan gum ⁽¹⁾ | 1.2 | |
| Sodium hyaluronate ⁽²⁾ | 1 | |
| PEG-40 hydrogenated castor oil ⁽³⁾ | 0.2 | |
| Clove extract | 0.4 | |
| (Eugenia caryophyllus bud oil) | | |
| Sodium bicarbonate ⁽⁴⁾ | 1.1 | (99.47% a.i.) |
| Common lady's mantle extract ⁽⁵⁾ | 5 | (>1.5% a.i.) |
| Sage bio water-glycerol extract 80 ⁽⁶⁾ | 5 | |
| Curcumin ⁽⁷⁾ | 0.0003 | (90% a.i.) |
| Calendula officinalis leaf extract ⁽⁸⁾ | 5 | |
| Panthenol ⁽⁹⁾ | 0.5 | |
| Avocado oil | 0.5 | (30-50% a.i.) |
| (Persea gratissima oil) ⁽¹⁰⁾ | | |
| Stevia rebaudiana extract ⁽¹¹⁾ | 0.5 | (>97% a.i.) |
| Xylitol ⁽¹²⁾ | 1 | (>98.5% a.i.) |

| | |
|---|---|
| Cetyl pyridinium chloride | 1-3% |
| Grapefruit seed extract | 33% |
| (Citrus paradisi peel extract) ⁽¹³⁾ | |
| Water | qs 100 |

a.i.: active ingredients
⁽¹⁾ Sold under the trade name Keltrol CG
⁽²⁾ Sold under the trade name Nutrihyl
⁽³⁾ Sold under the trade name Tagat CH 40,
⁽⁴⁾ Sold under the trade name Bicarbonate sodium ALIM E500
⁽⁵⁾ Sold under the trade name Alchemille extrait hydroalcoolique
⁽⁶⁾ Sold under the trade name Sauge Bio Extrait Hydroglycérine
⁽⁷⁾ Sold under the trade name Napture Col Yellow LC 112
⁽⁸⁾ Sold under the trade name Calendula Extrait Hydroglycérine 80
⁽⁹⁾ Sold under the trade name Dexpanthénol 75% Solution
⁽¹⁰⁾ Sold under the trade name Huile Avocat Hydrosoluble
⁽¹¹⁾ Sold under the trade name Extrait de Stevia 97% REB-A
⁽¹²⁾ Sold under the trade name Xivia C
⁽¹³⁾ Sold under the trade name EPP Sirop Agave Abio

### II. Procedure

A powder mixture composed of xanthan gum and sodium hyaluronate is introduced gradually into water, with vigorous stirring, until a uniform gel is obtained.

Then the PEG-40 hydrogenated castor oil, the clove extract and the sodium bicarbonate are introduced, and the mixture is stirred vigorously for 5 to 10 minutes between each addition, to give a uniform mixture.

The remainder of the ingredients are then incorporated, finishing with the cetylpyridinium chloride and the grapefruit seed extract, and the mixture is then stirred at moderate speed so as to limit the formation of foam.

A composition is obtained with a final pH of 7.3 to 7.7.

### III. Study of the Antimicrobial Protection of Composition (C) According to the Invention

In this example, the antimicrobial protection of a composition (C) according to the invention is evaluated according to the ISO 11930 standard.

This evaluation is based on the inoculation of composition (C) into five different strains of microorganisms (Pseudomonas aeruginosa, Staphylococcus aureus, Escherichia coli, Candida albicans, and Aspergillus brasiliensis) with calibrated inocula.

The number of surviving microorganisms is then measured at pre-defined time intervals over 28 days on enumeration agars.

The agars used for enumeration are tryptocasein soya agar (TSA) for the bacteria Pseudomonas aeruginosa, Staphylococcus aureus, and Escherichia coli, sabouraud dextrose agar (SDA) for Candida albicans, and potato dextrose agar (PDA) for Aspergillus brasiliensis.

For each time and each strain, the degree of reduction of the microorganisms is calculated and is compared with the minimum values required for the A or B evaluation criteria of the ISO 11930 standard.

### IV. Results

The degrees of reduction of microorganisms are indicated as percentages in the table below:

| | Degree of reduction of |
|---|---|
| Microorganisms | microorganisms (Tx) |
| Pseudomonas aeruginosa | Tx >99.9% of the inoculated |
| | bacteria at 7 days |
| Staphylococcus aureus | Tx >99.9% of the inoculated |
| | bacteria at 7 days |
| Escherichia coli | Tx >99.9% of the inoculated |
| | bacteria at 7 days |
| Candida albicans | Tx >99.9% of the inoculated |
| | yeasts at 7 days |
| Aspergillus brasiliensis | Tx >99% of the inoculated |
| | moulds at 14 days |

The degrees of reduction of microorganisms correspond to the criteria of profile A of the ISO 11930 standard.

Composition (C) according to the invention therefore meets the evaluation criteria of profile A with regard to antimicrobial protection in the ISO 11930 standard, and is therefore effective against Pseudomonas aeruginosa, Staphylococcus aureus, Escherichia coli, Candida albicans, and Aspergillus brasiliensis.

### Example III

In this example, the virucidal activity of the composition (C) according to the invention is studied, as described in example II, in accordance with the NF EN 14476 standard.

### I. Methodology

The virucidal activity of the composition is tested according to the methodology of the NF EN 14476 standard.

The virus strain tested is herpes virus type 1 (HSV1), cultured on VERO cells, at a temperature of 37° C. under 5% CO₂.

This evaluation takes place for contact times of 30, 45 and 60 seconds between the composition according to the invention and the viral suspension.

The titring is calculated according to the Spearman-Karber method.

### II. Results

Composition (C) according to the invention is effective after 45 seconds of contact with herpes virus type I at a temperature of 20° C. according to the methodology of the NF EN 14476 standard, with a degree of reduction of more than 99.99% of the virus.

### Example IV

In this example, the bactericidal activity of the composition (C) according to the invention is studied, as described in example II, on the Streptococcus mutans in accordance with the NF EN 13727 standard.

Results

Composition (C) according to the invention is effective after one minute on Streptococcus mutans at a temperature of 20° C. in accordance with the methodology of the NF EN 13727 standard, with a degree of reduction of more than 99.99% of the bacteria.

### Example V

### I. Preparation of Composition (D) According to the Invention

Composition (D) according to the invention is prepared in accordance with the procedure described below. The amounts are expressed as percentage by weight unless otherwise indicated.

| | Amounts (% by weight) | |
|---|---|---|
| Sodium hyaluronate ⁽¹⁾ | 1 | |
| PEG-40 hydrogenated castor oil ⁽²⁾ | 0.2 | |
| Clove extract | 0.4 | |
| (Eugenia caryophyllus bud oil) | | |
| Sodium bicarbonate ⁽³⁾ | 1.1 | (99.47% a.i.) |
| Common lady's mantle extract ⁽⁴⁾ | 5 | (>1.5% a.i.) |
| Sage bio water-glycerol extract 80 ⁽⁵⁾ | 5 | |
| Curcumin ⁽⁶⁾ | 0.0003 | (90% a.i.) |
| Calendula officinalis leaf extract ⁽⁷⁾ | 5 | |
| Panthenol ⁽⁸⁾ | 0.5 | |
| Avocado oil | 0.5 | (30-50% a.i.) |
| (Persea gratissima oil) ⁽⁹⁾ | | |
| Stevia rebaudiana extract ⁽¹⁰⁾ | 0.5 | (>97% a.i.) |
| Xylitol ⁽¹¹⁾ | 1 | (>98.5% a.i.) |

| | |
|---|---|
| Cetyl pyridinium chloride | 1-3% |
| Grapefruit seed extract | 33% |
| (Citrus paradisi peel extract) ⁽¹²⁾ | |
| Water | Qs 100 |

a.i.: active ingredients
⁽¹⁾ Sold under the trade name Nutrihyl
⁽²⁾ Sold under the trade name Tagat CH 40,
⁽³⁾ Sold under the trade name Bicarbonate sodium ALIM E500
⁽⁴⁾ Sold under the trade name Alchemille extrait hydroalcoolique
⁽⁵⁾ Sold under the trade name Sauge Bio Extrait Hydroglycérine
⁽⁶⁾ Sold under the trade name Napture Col Yellow LC 112
⁽⁷⁾ Sold under the trade name Calendula Extrait Hydroglycérine 80
⁽⁸⁾ Sold under the trade name Dexpanthénol 75% Solution
⁽⁹⁾ Sold under the trade name Huile Avocat Hydrosoluble
⁽¹⁰⁾ Sold under the trade name Extrait de Stevia 97% REB-A
⁽¹¹⁾ Sold under the trade name Xivia C
⁽¹²⁾ Sold under the trade name EPP Sirop Agave Abio

### II. Procedure

Sodium hyaluronate is introduced gradually into water, with vigorous stirring, until a uniform gel is obtained.

Then the PEG-40 hydrogenated castor oil, the clove extract and the sodium bicarbonate are introduced, and the mixture is stirred vigorously for 5 to 10 minutes between each addition, to give a uniform mixture.

The remainder of the ingredients are then incorporated, finishing with the cetylpyridinium chloride and the grapefruit seed extract, and the mixture is then stirred at moderate speed so as to limit the formation of foam.

A composition is obtained with a final pH of 7.3 to 7.7.

### III. Study of the Antimicrobial Protection of Composition (D) According to the Invention

The antimicrobial protection of composition (D) according to the invention is studied according to the EN 14562 standard.

More particularly, the antimicrobial activity on Candida albicans is studied.

Composition (D) according to the invention is effective after one minute of contact at a temperature of 20° C. according to the methodology of the NF EN 14562 standard, with a degree of reduction of more than 99.99% of yeasts.

### Example VI

In this example, the buccodental effects of composition (C) according to the invention, as described in example II, are studied on a panel of volunteers composed of 70 healthy males and females individuals, which are regular or occasional users of hygiene oral health products, aged between from 18 to 70 years, all of whom have sensitive gums and carry crown(s), bridge(s) and dental implants.

### I. Procedure

Composition (C) according to the invention is applied at home by each member of the panel, under the normal conditions of use, for a duration of 7 consecutive days.

0.1 ml of the composition is applied to the gums three times per day for a period of seven days. The composition is rubbed in and is not rinsed off.

A buccodental examination is carried by the same dentist for the whole of the panel at D1 (TO) before the 1st application of the composition, and at D7 after seven days of use.

The appraisal of the cosmetic qualities and of the efficacy of the composition according to the invention is recorded in a questionnaire completed by the panel of volunteers after the seven consecutive days of use.

The panel is made up of 70 persons aged from 18 to 70 years.

### II. Results

The percentages of volunteers who are satisfied according to the cosmetic and efficacy criteria for the composition are collated in the table below:

| | Percentage of |
|---|---|
| Evaluation criteria | volunteers satisfied |
| Composition is easy to apply | 94% |
| Film-forming texture allows the composition to | 71% |
| be maintained in the buccal cavity | |
| Composition suits me | 96% |
| Composition suitable for daily use | 97% |
| Composition gives no sensations of discomfort | 97% |
| during use | |
| Composition gives no sensations of discomfort | 99% |
| after use | |
| Composition is suitable for my type of gum | 99% |
| Composition is respectful of the balance of the | 100% |
| buccal cavity | |
| Composition made my gums stronger | 97% |
| Composition helps to alleviate my sensitive gums | 98% |
| Composition reduced the bleeding of my gums | 97% |
| Composition reduced the sensitivity of my teeth | 95% |
| in thermal contact (cold or hot) | |
| Immediate efficacy of the composition | 94% |
| Efficacy is lasting | 97% |

The composition according to the invention is found to exhibit excellent buccodental properties.

Thus the composition according to the present invention has a good buccodental acceptability.

### Example VII

In this example, the buccodental effects of composition (C) according to the invention, as described in example II, were studied by an external research and experimentation laboratory.

### I. Procedure

This study was carried out under the same conditions as the preceding study, in other words on a panel of volunteers composed of 20 healthy individuals under normal conditions of use.

The appraisal of the cosmetic qualities and the efficacy of the composition according to the invention are recorded in a questionnaire completed by the panel of volunteers after the seven consecutive days of use.

Furthermore, the clinical signs and the sensations of discomfort are reported directly by the participants during the study or are indicated in the daily monitoring report.

### II. Clinical Signs and Sensations of Discomfort Recorded

The clinical signs and the sensations of discomfort are recorded in the tables hereinafter:

| | Percentage of |
|---|---|
| | volunteers concerned |
| Clinical signs recorded | |
| Slight drying of the lips and slight tongue | 5% |
| depapillation | |
| Depapillation of the lateral edges of the tongue | 5% |
| Slight colouration of the tongue | 5% |
| Sensations of discomfort recorded | |
| Substantial adverse effect on sense of taste after | 5% |
| each application for an hour | |
| Slight adverse effect on the sense of taste after | 5% |
| each application for an hour, with substantial | |
| hypersalivation for 5 to 10 minutes after | |
| application of the composition for 30 minutes | |
| Moderate dryness of the gums after each | 5% |
| application, permanent | |
| Substantial adverse effect on the sense of taste, | 5% |
| and moderate change in the breath after each | |
| application until rinsing | |
| Moderate adverse effect on the sense of taste | 5% |
| after each application for 1 to 2 hours | |
| Moderate hypersalivation after each application | 5% |
| for 5 to 10 minutes | |
| Bitterness in the mouth | — |

It is found that the sensations of discomfort and the negative clinical signs recorded are low for the composition according to the invention.

### III. Criteria for Buccodental Evaluation of the Composition

| | Percentage of |
|---|---|
| Evaluation criteria | volunteers satisfied |
| Film-forming texture allows the composition to | 85% |
| be kept in the buccal cavity | |
| Composition is suitable for daily use | 65% |
| Composition does not give sensations of discomfort | 75% |
| during use | |
| Composition does not give sensations of discomfort | 85% |
| after use | |
| Composition is suitable for my type of gums | 75% |
| Composition is respectful of the balance of the | 80% |
| buccal cavity | |
| Composition helps with the alleviation of my | 65% |
| sensitive gums | |
| Composition reduced the bleeding of my gums | 70% |
| Composition reduced the sensitivity of my teeth | 60% |
| on thermal contact (cold or hot) | |
| Composition reduces the sensation of halitosis | 75% |

It is found that the composition according to the invention exhibits excellent buccodental properties overall.

1. Composition comprising at least one grapefruit seed extract, at least one lady's mantle leaf extract, at least one stevia extract, and at least curcumin.

2. Composition according to claim 1, characterized in that the lady's mantle leaf extract is an extract of common lady's mantle leaf.

3. Composition according to claim 1, characterized in that the stevia corresponds to Stevia rebaudiana.

4. Composition according to claim 1, characterized in that it is in gel form and comprises at least one, preferably nonionic, gelling agent.

5. Composition according to claim 4, characterized in that the nonionic gelling agent is selected from polysaccharides, and preferably a xanthan gum.

6. Composition according to claim 1, characterized in that it further comprises sodium hyaluronate.

7. Composition according to claim 1, characterized in that it is in the form of a medical device.

8. Composition according to claim 1 for use in the therapeutic sector.

9. Composition according to claim 1 for use for the treatment of complaints of the buccal cavity.

10. Composition according to claim 9, characterized in that the complaints of the buccal cavity are selected from bleeding, lesions of the buccal mucosa and/or of the tongue, inflammatory disorders other than lesions, dental eruptions, gingival sensitivity, dental sensitivity, halitosis, and bacterial, fungal, and viral infections.

11. Composition according to claim 1 for use in the treatment of mouth ulcers.

12. Composition according to claim 1 for use in the treatment of thrush.

13. Composition according to claim 1 for use in the treatment of complaints caused by bacteria selected from Streptococcus mutans, Prevotella melaninogenica, Pseudomonas aeruginosa, Staphylococcus aureus, and Escherichia coli.

14. Composition according to claim 1 for use in diminishing or alleviating the pain caused by one or more buccal cavity complaints selected from bleeding, lesions of the buccal mucosa and/or of the tongue, inflammatory disorders other than lesions, dental eruptions, gingival sensitivity, dental sensitivity, halitosis, and bacterial, fungal, and viral infections.

15. Composition according to claim 1 for use for the treatment of complaints other than those of the buccal cavity.

16. Composition according to claim 1, characterized in that it is in the form of a cosmetic composition.

17. The composition according to claim 8, characterized in that it is suitable for application to the buccal cavity surface or surfaces to be treated.

18. Cosmetic use of the composition as defined according to claim 1 for preserving the resident flora of the buccal cavity.
